(19) United States
(12) Patent Application Publication
(10) Pub. No.: US 2026/0207191 A1
(43) Pub. Date: Jul. 23, 2026

(54) MEDICAL NEEDLE ASSEMBLY

(71) Applicants: Shih-Hsien WANG, Putz, Chiayi County (TW); Dong-Ru HO, Putz, Chiayi County (TW)

(72) Inventors: Shih-Hsien WANG, Putz, Chiayi County (TW); Dong-Ru HO, Putz, Chiayi County (TW)

(21) Appl. No.: 19/141,879

(22) PCT Filed: Aug. 19, 2024
(86) PCT No.: PCT/CN2024/113017

§ 371 Date: Jun. 20, 2025

Related U.S. Application Data
(60) Provisional application No. 63/535,048, filed on Aug. 28, 2023.

(51) Int. Cl. A61B 17/04; A61B 17/06; A61B 17/34

(52) U.S. Cl. CPC A61B 17/0469; A61B 17/34; A61B 2017/061

(57) ABSTRACT

Provided in the present invention is a medical needle assembly, comprising an outer tubular needle, which comprises an outer tubular needle body that is hollow inside through two ends and defines a first inner diameter and a first length; a puncture needle, which comprises a puncture needle body that defines a first outer diameter and a second length; and an inner tubular needle that is hollow inside through two ends and defines a second outer diameter and a third length, the first inner diameter being greater than the first outer diameter and the second outer diameter, and the first length being less than the second length and the third length.

### FIELD

The present invention relates to a medical instrument, specifically, a medical needle assembly.

### BACKGROUND

Suturing is a commonly used medical procedure. The primary purposes of performing suturing include: restoring the original function of an organ or tissue, removing foreign objects, or placing medical devices. Understandably, the suturing method and process often directly affect the patient's postoperative recovery.

During the suturing process, needles are typically used. For instance, when a patient suffers a wound on the body surface, a physician manipulates a needle carrying a suture thread to pass through the wound, thereby enabling closure of the wound. A common practice involves using a curved suture needle as the needle instrument, which is held by a needle holder to guide the suture thread through the wound and close it by forming knots. When a perforation occurs inside the patient's body, the patient often expects minimal postoperative scarring and pain. Therefore, in consideration of such needs, physicians may adjust the medical approach, including selecting appropriate medical needles and applying compatible suturing methods, to achieve the desired outcomes of minimal scarring and reduced pain, thus meeting the patient's expectations.

### SUMMERY

To improve the prior art, the first object of the present invention is to provide a medical needle assembly, comprising: an outer tubular needle, comprising: an outer tubular needle body having an hollow interior extending through both ends, wherein the outer tubular needle body is defined as having a first inner diameter, a first length, and a first opening arranged on an end of the outer tubular needle body; a puncturing needle, comprising: a puncturing needle body with both ends, having a first outer diameter and a second length; and an inner tubular needle, comprising: an inner tubular needle body having an hollow interior extending through both ends, wherein the inner tubular needle body is defined as a second outer diameter, a third length and a second opening arranged on an end of the inner tubular needle body; wherein the first inner diameter is larger than the first outer diameter and the second outer diameter, and the first length is less than the second length and the third length. In that case, the puncturing needle body can insert into the outer tubular needle body, the inner tubular needle body can insert into the outer tubular needle body, and including:
(A) inserting an end of the puncturing needle body into the interior of the outer tubular needle body through the first opening, protruding the end of the puncturing needle body from the other end of the outer tubular needle body to contact a wound, and separating the puncturing needle body and the outer tubular needle body;
(B) inserting an end of a first suture thread into the interior of the inner tubular needle body through the second opening, protruding the end of the first suture thread from the other end of the inner tubular needle body, moving the end of the first suture thread close to the other end of the first suture thread outside the interior of the inner tubular needle body, and forming a first inflection point on a part of the first suture thread near to the other end of the inner tubular needle body; and
(C) maintaining the first inflection point, inserting the other end of the inner tubular needle body into the outer tubular needle body through the first opening, protruding the first inflection point and the other end of the inner tubular needle body from the other end of the outer tubular needle body, and allowing the first inflection point to form a first loop by advancing the other end of the first suture thread.

In some preferred embodiments, the outer tubular needle further comprises: a first limiting portion arranged around the first opening; and a first needle tip portion arranged on the other end of the outer tubular needle body.

In some preferred embodiments, the puncturing needle further comprises: a second needle tip portion arranged on an end of the puncturing needle body; and a first holding portion arranged on the other end of the puncturing needle body.

In some preferred embodiments, the inner tubular needle further comprises: a second holding portion arranged around the second opening; and a third needle tip portion arranged on the other end of the inner tubular needle portion.

In some preferred embodiments, the first length ranges from 4 to 10 cm, the second length ranges from 5 to 11 cm, and the third length ranges from 6.5 to 12.5 cm.

In some preferred embodiments, the first inner diameter ranges from 2 to 5 mm, the first outer diameter ranges from 1.5 to 4 mm, the second outer diameter ranges from 1.5 to 3 mm.

In some preferred embodiments, the first outer diameter may be identical to or different from the second outer diameter.

In some preferred embodiments, the first limiting portion has a first interface arranged between the first limiting portion and the first opening, wherein the interior of the first interface has a groove structure, an opening of the groove structure is arranged on an end of the first limiting portion relatively distant from the first opening, and a bottom of the groove structure connects to the first opening.

In some preferred embodiments, the second holding portion has a second interface arranged between the second holding portion and the second opening, wherein the exterior of the second interface has a protruding structure extending toward the inner tubular needle body, a surface of the protruding structure has one or more spline grooves parallel to the inner tubular needle body.

In some preferred embodiments, the first interface is complementary to the second interface, and the one or more spline grooves are configured to accommodate the first suture thread, thereby, in (C): maintaining the first inflection point, inserting the other end of the inner tubular needle body into the outer tubular needle body through the first opening, allowing to combine the first interface and the second interface, to accommodate the end of the first suture thread in the one or more spline grooves and to protrude the other end of the inner tubular needle body with the first inflection point from the other end of the outer tubular needle body, subsequently, advancing the other end of the first suture thread, thereby allowing the first inflection point to form the first loop.

In some preferred embodiments, another object of the present invention is to provide a method of painless hernia ligation, comprising:
step (A1): obtaining a medical needle assembly as mentioned above;
step (A2): combining the puncturing needle to the outer tubular needle;
step (A3): puncturing a body surface of a patient using the combination of the puncturing needle and the outer tubular needle, and maintaining the outer tubular needle in position over a hernial orifice;
step (A4): combining a first suture thread to the inner tubular needle;
step (A5): combining the inner tubular needle with the first suture thread to the outer tubular needle, and allowing an end of the first suture thread to reach an underside of a first side of the hernial orifice;
step (A6): forming a first loop on the underside of the first part;
step (A7): withdrawing the inner tubular needle from the outer tubular needle, and reinserting the inner tubular needle into the outer tubular needle;
step (A8): combining a second suture thread to the inner tubular needle, and allowing an end of the second suture thread to reach an underside of a second side of the hernial orifice;
step (A9): combining the first suture thread and the second suture thread to perform the ligation.

In summary, the medical needle assembly provided by the present invention is suitable for wound suturing and for performing the method for painless hernia ligation, wherein in the step (A3), the outer tubular is maintained in position over the hernial orifice, thereby preventing from the requirement of multiple insertion and forming a thread channel, subsequently, in the step (A9), the thread channel can be used to allow a knot formed by the first suture thread and the second suture thread to pass through from the body surface into the wound, and to avoid a nerve between the body surface and the hernial orifice from being bound by the knot, thereby achieving the effect of the painless hernia ligation.

### BRIEF DESCRIPTION OF THE DRAWINGS

FIG. 1 is a schematic diagram of the medical needle assembly.

FIG. 2 is a schematic diagram of the outer tubular needle.

FIG. 3 is a schematic diagram of the first limiting portion.

FIG. 4 is a schematic diagram of the puncturing needle.

FIG. 5 is a sectional diagram of the first holding portion.

FIG. 6 is a schematic diagram of the inner tubular needle.

FIG. 7 is a schematic diagram of the second interface.

FIG. 8 is a schematic diagram for illustrating the combination of the puncturing needle and the outer tubular needle.

FIG. 9 is schematic diagram for illustrating the combination of the inner tubular needle and the outer tubular needle.

FIG. 10 is a first schematic diagram of a method of painless hernia ligation.

FIG. 11 is a second schematic diagram of a method of painless hernia ligation.

FIG. 12 is a third schematic diagram a method of painless hernia ligation.

FIG. 13 is a fourth schematic diagram a method of painless hernia ligation.

FIG. 14 is a fifth schematic diagram a method of painless hernia ligation.

FIG. 15 is a sixth schematic diagram a method of painless hernia ligation.

FIG. 16 is a seventh schematic diagram a method of painless hernia ligation.

FIG. 17 is an eighth schematic diagram a method of painless hernia ligation.

FIG. 18 is a schematic diagram of a method of painless hernia ligation using mesh.

### DETAILED DESCRIPTION

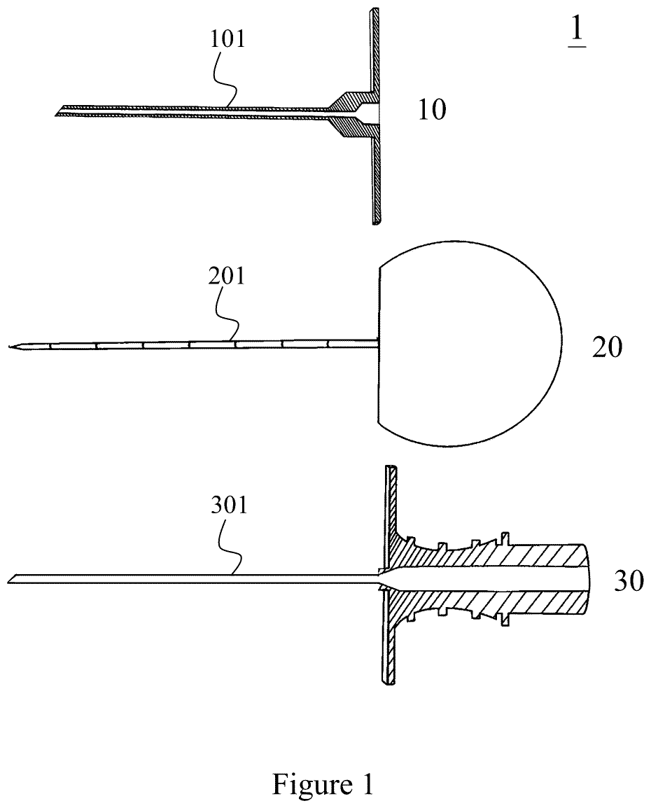

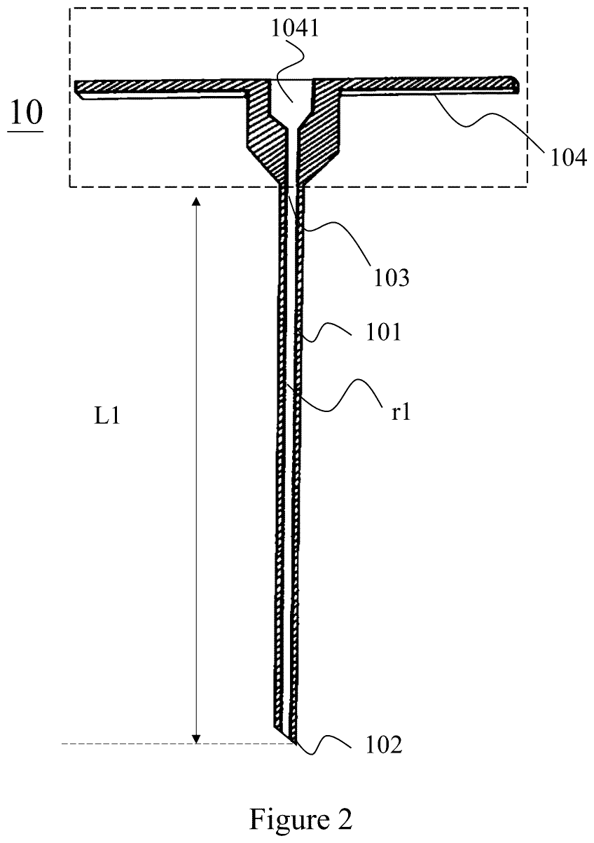

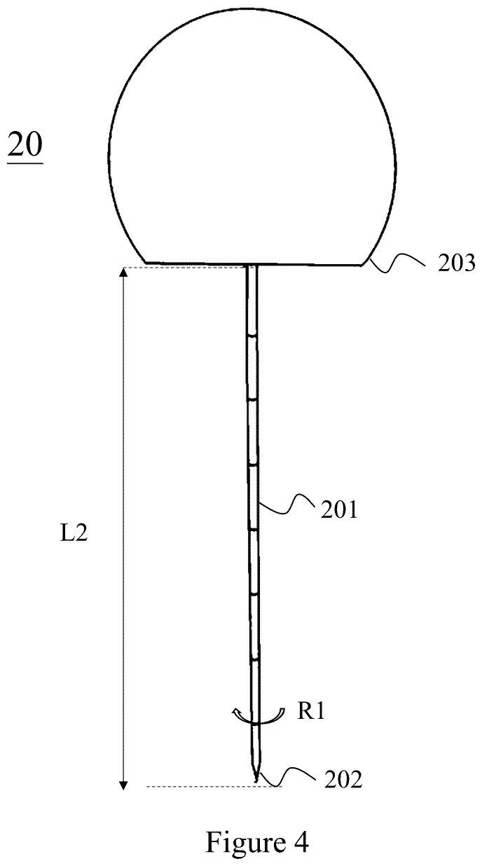

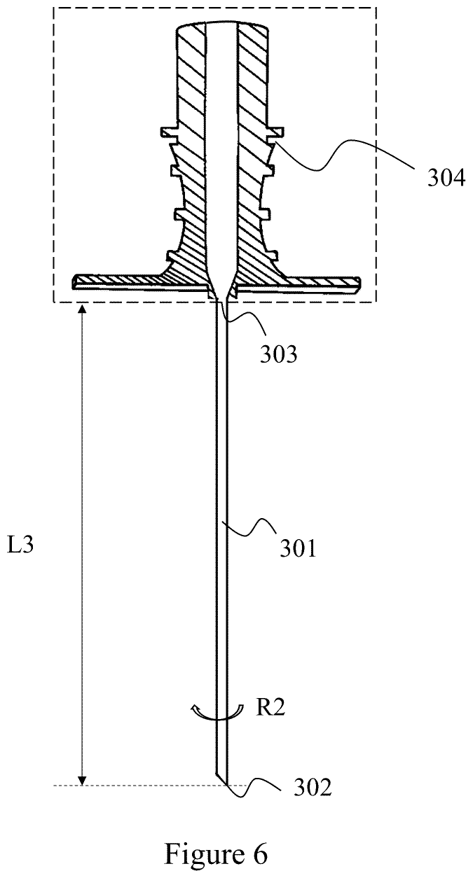

With reference to FIG. 1, FIG. 2, FIG. 4, and FIG. 6, FIG. 1 is a schematic diagram of a medical needle assembly, FIG. 2 is a schematic diagram of the outer tubular needle, FIG. 4 is a schematic diagram of the puncturing needle, FIG. 6 is a schematic diagram of the inner tubular needle; In some preferred embodiments, a medical needle assembly 1 comprising: an outer tubular needle 10, comprising: an outer tubular needle body 101 having an hollow interior extending through both ends, wherein the outer tubular needle body 101 is defined as having a first inner diameter (r1), a first length (L1), and a first opening 103 arranged on an end of the outer tubular needle body 101; a puncturing needle 20, comprising: a puncturing needle body 201 with both ends, having a first outer diameter (R1) and a second length (L2); and an inner tubular needle 30, comprising: an inner tubular needle body 301 having an interior extending through both ends, wherein the inner tubular needle body 301 is defined as having a second outer diameter (R2), a third length (L3) and a second opening 303 arranged on an end of the inner tubular needle body 301; wherein the first inner diameter (r1) is larger than the first outer diameter (R1) and the second outer diameter (R2), and the first length (L1) is less than the second length (L2) and the third length (L3); thereby:
(A) inserting an end of the puncturing needle body 201 into the hollow interior of the outer tubular needle body 101 through the first opening 103, protruding the end of the puncturing needle body 201 from the other end of the outer tubular needle body 101 to contact a wound, and separating the puncturing needle body 201 and the outer tubular needle body 101;
(B) inserting an end of a first suture thread S1 into the hollow interior of the inner tubular needle 301 through the second opening 303, protruding the end of the first suture thread S1 from the other end of the inner tubular needle 301, moving the end of the first suture thread S1 close to the other end of the first suture thread S1 outside the hollow interior of the inner tubular needle 301, and forming a first inflection point C1 on a part of the first suture thread S1 near to the other end of the inner tubular needle 301; and
(C) maintaining the first inflection point C1, inserting the other end of the inner tubular needle 301 into the outer tubular needle body 101 through the first opening 103, protruding the first inflection point C1 and the other end of the inner tubular needle 301 from the other end of the outer tubular needle body 101, and allowing the first inflection point C1 to form a first loop O1 by advancing the other end of the first suture thread S1.

With reference to FIG. 2, in some preferred embodiments, the outer tubular needle 10 further comprises: a first limiting portion 104 arranged around the first opening 103; and a first needle tip portion 102 arranged on the other end of the outer tubular needle body 101.

In some preferred embodiments, the first limiting portion 104 is detachably arranged around the first opening 103.

With reference to FIG. 2, in some preferred embodiments, the first limiting portion 104 has a first interface 1041 arranged between the first limiting portion 104 and the first opening 103.

In some preferred embodiments, the interior of the first interface 1041 has a groove structure, an opening of the groove structure is arranged on an end of the first limiting portion 104 relatively distant from the first opening 103, a bottom of the groove structure connects to the first opening 103.

In some preferred embodiments, the first limiting portion 104 has an outwardly extending single-arm structure or an outwardly extending dual-arm structure.

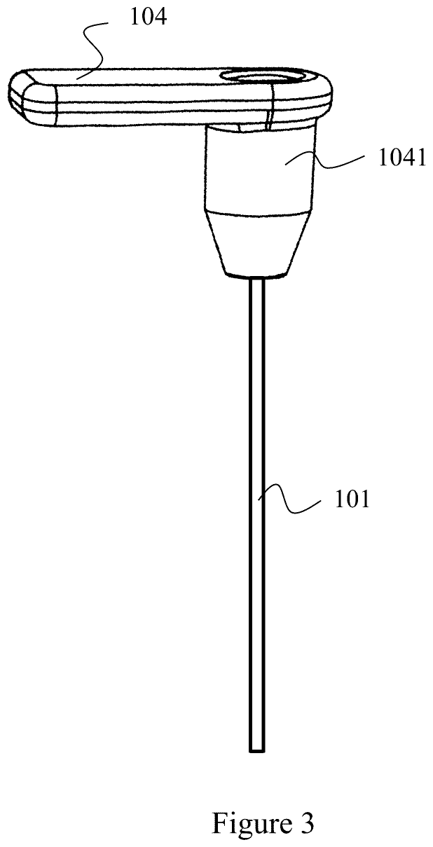

With reference to FIG. 3, FIG. 3 is a schematic diagram of the first limiting portion 104; in some preferred embodiment, the first limiting portion 104 has the outwardly extending single-arm structure, and has the first interface 1041 connected to the first opening 103.

With reference to FIG. 4, in some preferred embodiments, the puncturing needle 20 further comprises: a second needle tip portion 202 arranged on an end of the puncturing needle body 201; and a first holding portion 203 arranged on the other end of the puncturing needle body 201.

With reference to FIG. 4, in some embodiments, the first holding portion 203 may have different structure, for example, the first holding portion 203 may be a handle structure, a spherical structure, or a columnar structure.

In some preferred embodiments, the first holding portion 203 is detachably arranged on the other end of the puncturing needle body 201.

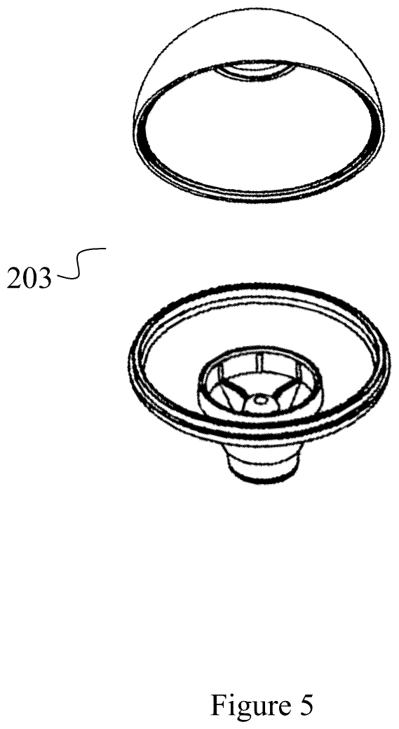

With reference to FIG. 5, FIG. 5 is a sectional diagram of the first holding portion, in some preferred embodiment, the first holding portion 203 is a hollow spherical structure.

In some preferred embodiment, an outer periphery part of the first holding portion 203 is larger than the perimeter of the first opening 103, or the outer periphery part of the first holding portion 203 is larger than the first inner diameter (r1).

In some preferred embodiment, the first holding portion 203 is configured as a handle, the outer periphery part of the first holding portion 203 is larger than the perimeter of the first opening 103, or the outer periphery part of the first holding portion 203 is larger than the first inner diameter (r1).

With reference to FIG. 6, in some preferred embodiments, the inner tubular needle 30 further comprises: a second holding portion 304 arranged around the second opening 303; and a third needle tip portion 302 arranged on the other end of the inner tubular needle body 301.

With reference to FIG. 6, in some preferred embodiments, the second holding portion 304 may have different structure, for example, the second holding portion 304 may be a handle structure, a spherical structure, or a columnar structure.

In some preferred embodiments, the first holding portion 203 is detachably arranged around the second opening 303.

In some preferred embodiments, the second holding portion 304 is configured as a handle, the outer periphery part of the second holding portion 304 is larger than the perimeter of the first opening 103, or the outer periphery part of the second holding portion 304 is larger than the first inner diameter (r1).

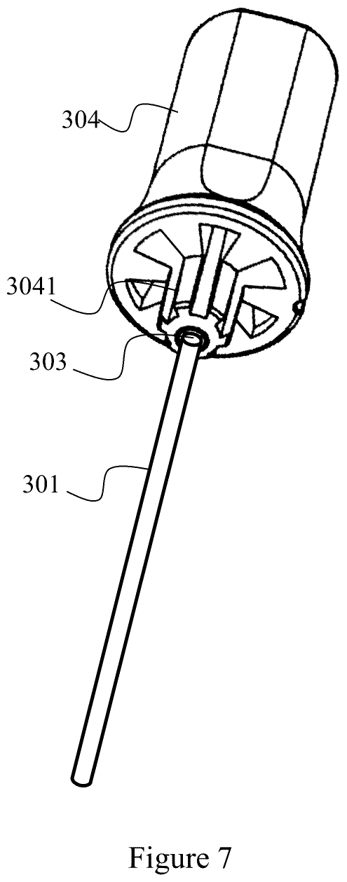

With reference to FIG. 7, FIG. 7 is a schematic diagram of the second interface; in some preferred embodiments, the second holding portion 304 has a second interface 3041 arranged between the second holding portion 304 and the second opening 303.

In some preferred embodiments, the exterior of the second interface 3041 has a protruding structure extending toward the inner tubular needle 301, a surface of the protruding structure has one or more spline grooves parallel to the inner tubular needle 301.

In some preferred embodiments, the first interface 1041 stably connects to the second interface 3041, wherein the first interface 1041, configured as a groove structure, is complementary to the second interface 3041, configured as a protruding structure.

With reference to FIG. 2 and FIG. 7, in some preferred embodiments, when the inner tubular needle 301 is inserted into the outer tubular needle body 101, the second interface 3041 and the first interface 1041 are brought into engagement; the one or more spline grooves parallels to the outer tubular needle body 101, and are allowed to accommodate a suture thread, wherein the suture thread can pass through the space between the inner tubular needle 301, and the outer tubular needle body 101.

In some preferred embodiments, the second holding portion 304 has an outwardly extending single-arm structure or an outwardly extending dual-arm structure.

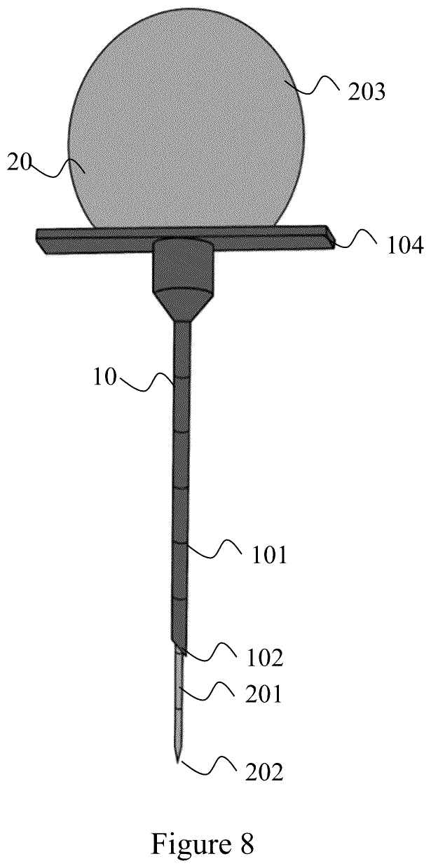

With reference to FIG. 8, FIG. 8 is a schematic diagram for illustrating the combination of the puncturing needle and the outer tubular needle; in some embodiment, the puncturing needle 20 can insert into the outer tubular needle 10 by its second needle tip portion 202 through the first opening 103, wherein the puncturing needle body 201 can be inserted into the outer tubular needle body 101, since the first inner diameter (r1) of the outer tubular needle body 101 is larger than the first outer diameter (R1) of the puncturing needle body 201; furthermore, the puncturing needle body 201 can protrude from the outer tubular needle body 101, or at least the second needle tip portion 202 can protrude from the outer tubular needle body 101, since the first length (L1) is less than the second length (L2).

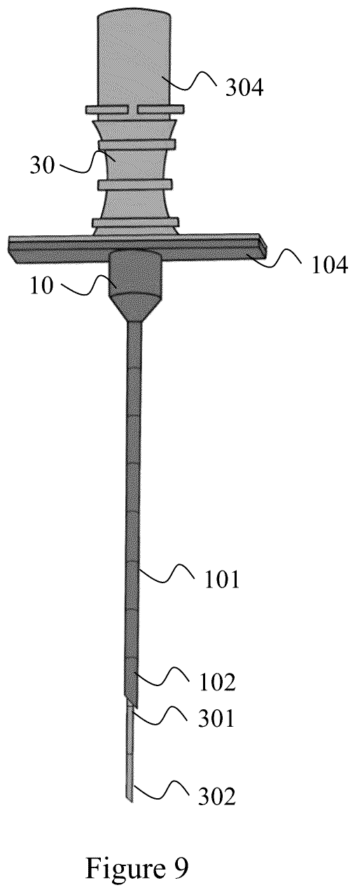

With reference to FIG. 9, FIG. 9 is schematic diagram for illustrating the combination of the inner tubular needle and the outer tubular needle; in some embodiment, the inner tubular needle 30 can insert into the outer tubular needle 10 by its third needle tip portion 302 through the first opening 103, wherein the inner tubular needle 301 can be inserted into the outer tubular needle body 101, since the first inner diameter (r1) of the outer tubular needle 10 is larger than the second outer diameter (R2) of the inner tubular needle 301; furthermore, the inner tubular needle 301 can protrude from the outer tubular needle body 101, or at least the third needle tip portion 302 can protrude from the outer tubular needle body 101, since the first length (L1) is less than the third length (L3).

With reference to FIG. 8, conceivably, the puncturing needle body 201 cannot be further advanced toward the second needle tip portion 202, since the outer periphery part of the first holding portion 203 is larger than the inner diameter of the first opening 103 or the outer periphery part of the first holding portion 203 is larger than the first inner diameter (r1), when the puncturing needle 20 inserts into the outer tubular needle 10 and the first opening 103 contacts the first holding portion 203.

In some preferred embodiments, the first opening 103 is closely fitted to the first holding portion 203, or the first limiting portion 104 is closely fitted to the first holding portion 203, when the puncturing needle 20 inserts into the outer tubular needle 10 and the first opening 103 contacts the first holding portion 203.

With reference to FIG. 9, similarly, the inner tubular needle body 301 cannot be further advanced toward the third needle tip portion 302, since the outer periphery part of the second holding portion 304 is larger than the inner diameter of the first opening 103 or the outer periphery part of the second holding portion 304 is larger than the first inner diameter (r1), when the inner tubular needle 30 inserts into the outer tubular needle 10 and the first opening 103 contacts the second holding portion 304.

In some preferred embodiments, the first opening 103 is closely fitted to the second holding portion 304, or the first limiting portion 104 is closely fitted to the second holding portion 304, when the inner tubular needle 30 inserts into the outer tubular needle 10 and the first opening 103 contacts the second holding portion 304.

In some preferred embodiments, the first length (L1) ranges from 4 to 10 cm, the second length (L2) ranges from 5 to 11 cm, and the third length (L3) ranges from 6.5 to 12.5 cm.

In some preferred embodiments, in a first specification, the first length (L1) is 4 cm, the second length (L2) is 5 cm, and the third length (L3) is 6.5 cm.

In some preferred embodiments, in a second specification, the first length (L1) is 7 cm, the second length (L2) is 8 cm, and the third length (L3) is 9.5 cm.

In some preferred embodiments, in a third specification, the first length (L1) is 10 cm, the second length (L2) is 11 cm, and the third length (L3) is 12.5 cm.

In some preferred embodiments, the second length (L2) may be identical or different from the third length (L3), and is not limited to these.

In some preferred embodiments, accordingly, under the condition that the first length (L1) is less than the second length (L2) and the third length (L3), the first length (L1), the second length (L2), or the third length (L3) are not limited, and are adjustable according to the application scenario. For example, the first specification, the second specification and the third specification can be selected for performing an abdomen-related surgery on a patient, based on the thickness of the abdominal fat layer.

In some preferred embodiments, the first inner diameter (r1) ranges from 2 to 5 mm.

In some preferred embodiments, specifically, the first inner diameter (r1) is 2.5 or 3.5 mm.

In some preferred embodiments, the first outer diameter (R1) ranges from 1.5 to 4.0 mm.

In some preferred embodiments, specifically, the first outer diameter (R1) is 3.0 mm.

In some preferred embodiments, the second outer diameter (R2) ranges from 1.5 to 3.5 mm.

In some preferred embodiments, specifically, the second outer diameter (R2) is 2.36 mm.

In some preferred embodiments, the first outer diameter (R1) is identical to or is different from the second outer diameter (R2).

In some preferred embodiments, understandably, under the condition that the first inner diameter (r1) is larger than the first outer diameter (R1) and the second outer diameter (R2), the first inner diameter (r1), the first outer diameter (R1), or the second outer diameter (R2) are not limited, and are adjustable according to the application scenario. For example, in a scenario where the puncturing needle body 201 or the inner tubular needle 301 is inserted into the outer tubular needle body 101, the ease of sliding between the puncturing needle body 201 and the outer tubular needle body 101, or the ease of sliding between the inner tubular needle 301 between the outer tubular needle body 101, will decrease if the first outer diameter (R1) or the second outer diameter (R2) is close to the first inner diameter (r1).

In some preferred embodiments, it is further understood that, the difference between the first inner diameter (r1) and the second outer diameter (R2) should be at least the total outer diameter of two suture threads, wherein the outer diameter of the suture thread is not limited, and can be decided based on the difference between the first inner diameter (r1) and the second outer diameter (R2).

In some preferred embodiments, the outer tubular needle 10, the puncturing needle 20, or the inner tubular needle 30 can be made of a material tolerant to sterilization or a reusable material; further, the outer tubular needle 10, the puncturing needle 20, or the inner tubular needle 30 can be made of a material intolerant to sterilization or a non-reusable material.

In some preferred embodiments, the outer tubular needle 10, the puncturing needle 20, or the inner tubular needle 30 is made of a medical plastic, the medical plastic comprises: polypropylene (PP), polyvinyl chloride (PVC), polyethylene (PE), plyetheretherketone (PEEK), polystyrene (PS), polycarbonate (PC), polytetrafluoroethene (PTFE), or acrylonitrile butadiene styrene (ABS).

In some preferred embodiments, the outer tubular needle 10, the puncturing needle 20, or the inner tubular needle 30 is made of a medical alloy, the medical alloy comprises: ferrochrome (iron chromium alloy), titanium alloy, nickel-cobalt alloy.

The following provides a first embodiment of the present invention, including performing (A) a method of painless hernia ligation by using the aforementioned medical needle assembly 1.

For example, the “upper part”, “upward”, “lower part”, and “downward” referred to herein is used to describe the relative position of a body surface 20 and a hernial orifice 231, wherein the body surface 20 refers to a body surface 20 of a patient, “the upper part of the body surface 20” indicates the position outside the patient's body or the position where the physician or the surgeon is located, “the lower part of the body surface 20” indicates the position inside the patient's body or the direction toward the position of the hernial orifice 231.

Furthermore, “the lower part” of the body surface of the patient sequentially comprises: epidermal layer 21, fascial layer 22, peritoneal layer 23, and neural layer between the epidermal layer 21 and the peritoneal layer 23, wherein the hernial orifice 231 is located on the peritoneal layer 23.

It is understood that a first side is located on the opposite side of a second side, with both parts correspondingly formed on the hernial orifice 231.

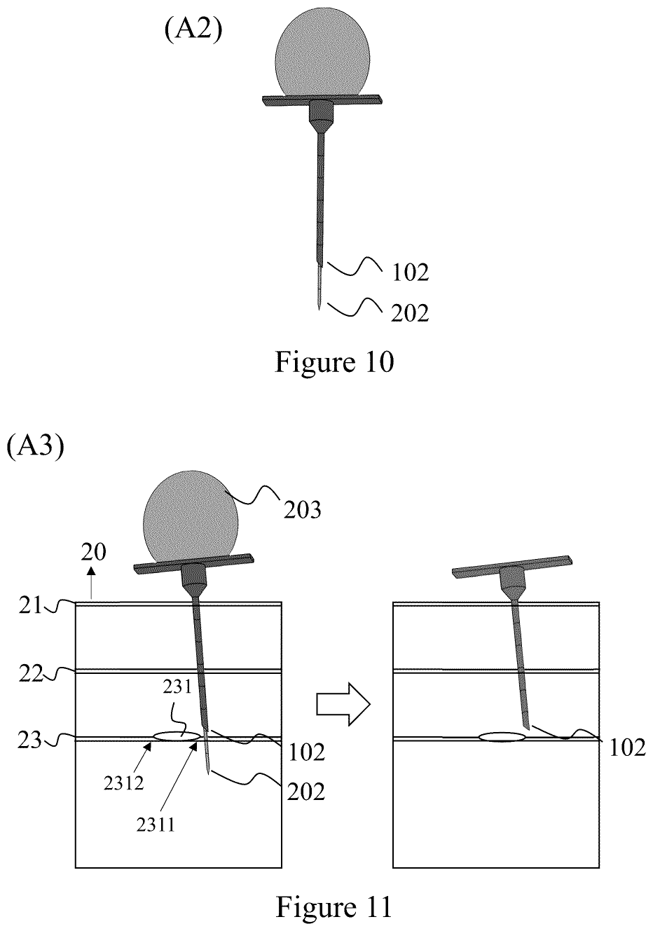

With reference to FIG. 10 to FIG. 17, FIG. 10 to FIG. 17 are schematic diagrams illustrating the method of painless hernia ligation. In some preferred embodiments, a method of painless hernia ligation (A) comprises:
Step (A1): obtaining a medical needle assembly 1 as mentioned above;

With reference to FIG. 10, step (A2): combining the puncturing needle 20 to the outer tubular needle 10: inserting the second needle tip portion 202 into the first opening 103, and allowing the second needle tip portion 202 to protrude from the first needle tip portion 102.

With reference to FIG. 11, step (A3): maintaining the outer tubular needle 10 at the location of the body surface 20 corresponding to the hernial orifice 231: using the puncturing needle 20 and the outer tubular needle 10 to penetrate the position on body surface 20 corresponding to the projection of hernial orifice 231 by holding the first holding portion 203, and to allow the second needle tip portion 202 to penetrate a first side of the hernial orifice 2311 to reach the lower part of the first side; removing the puncturing needle 20 from the first opening 103, maintaining the outer tubular needle 10, and maintaining the first needle tip portion 102 near to the first side of the hernial orifice 2311.

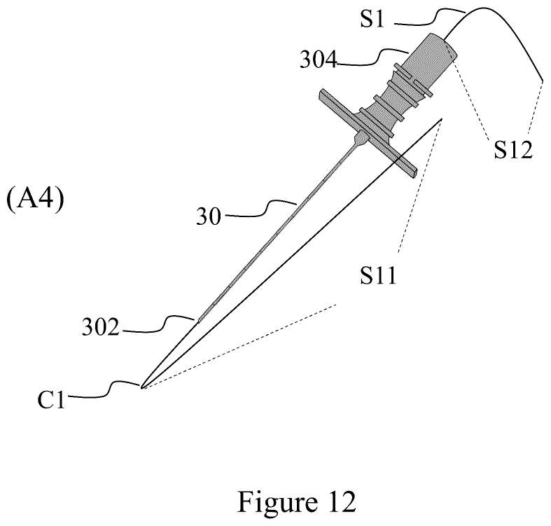

With reference to FIG. 12, step (A4): combining the first suture thread S1 to the inner tubular needle 30: inserting the first suture thread S1 into the second opening 303, and protruding the first suture thread S1 from the third needle tip portion 302, wherein the part of the first suture thread S1 protruding from the third needle tip portion 302 is defined as a first thread segment S11, and the part of the first suture thread S1 that does not insert into the inner tubular needle body 301 is defined as a second thread segment S12; and allowing the first thread segment S11 to get close to the second thread segment S12, thereby forming a first inflection point C1 on the first thread segment S11 outside the third needle tip portion 302.

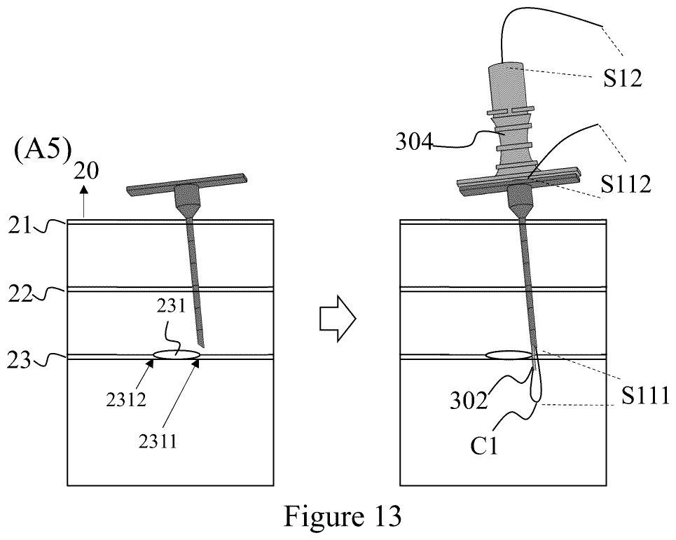

With reference to FIG. 13, step (A5): combining the first suture thread S1 and the inner tubular needle 30 to the outer tubular needle 10: inserting the third needle tip portion 302 into the first opening 103 accompanied by the first inflection point C1, and protruding the third needle tip portion 302 from the first needle portion. Understandably, the third needle tip portion 302 is passed through the first side of the hernial orifice 2311, and the first inflection point C1 is reached the lower part of the first side of the hernial orifice 2311. Herein, the first thread segment S11 is further defined as a first inside segment S111, which is extended from the third tip portion and is inserted into the first needle portion, and a first outside segment S112, which is extended from the gap between the first limiting portion 104 and the second holding portion 304 or is extended from the gap the first opening 103 and the second holding portion 304. It is further understood that, under the condition that the outer tubular needle 10 tightly combines to the inner tubular needle 30, which can be referred to that the first limiting portion 104 is tightly combined to the second holding portion 304 or the first opening 103 is tightly combined to the second holding portion 304, the length of the first outside segment S112 is controlled by compression. At this time, if the second thread segment S12 is advanced, the length of first inside segment S111 will increase, and the length of the first outside segment S112 will not increase.

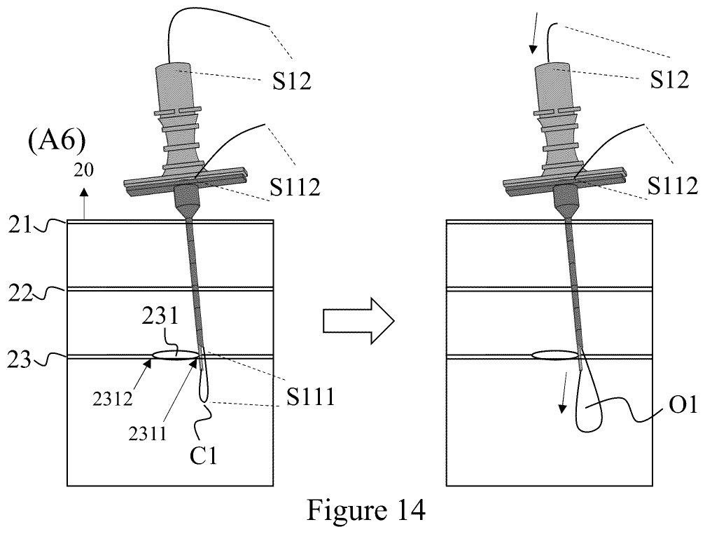

With reference to FIG. 14, step (A6): forming a first loop O1 at the lower part of the first side of the hernial orifice 2311: maintaining the tight combination between the first limiting portion 104 and the second holding portion 304 or maintaining the tight combination between the first opening 103 and the second holding portion 304, advancing the second thread segment S12 to increase the length of the first inside segment S111, thereby forming a first loop O1 at the lower part of the first side of the hernial orifice 2311.

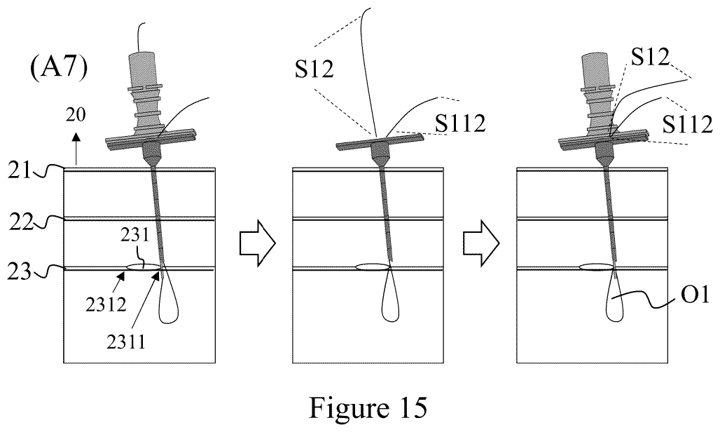

With reference to FIG. 15, step (A7): removing the inner tubular needle 30 from the outer tubular needle 10, and subsequently recombining the inner tubular needle 30 and the outer tubular needle 10: withdrawing the inner needle through the first opening 103, maintaining the first loop O1 at the lower part of the first side of the hernial orifice 2311, and recombining the inner tubular needle 30 to the outer tubular needle 10, that is to say, the third needle tip portion 302 protrudes from the first needle tip portion 102 through the first opening 103. Herein, the second thread segment S12 and the first outside segment S112 are both extended from the space between the first limiting portion 104 and the second holding portion 304, and the first loop O1 is extended from the first needle tip portion 102.

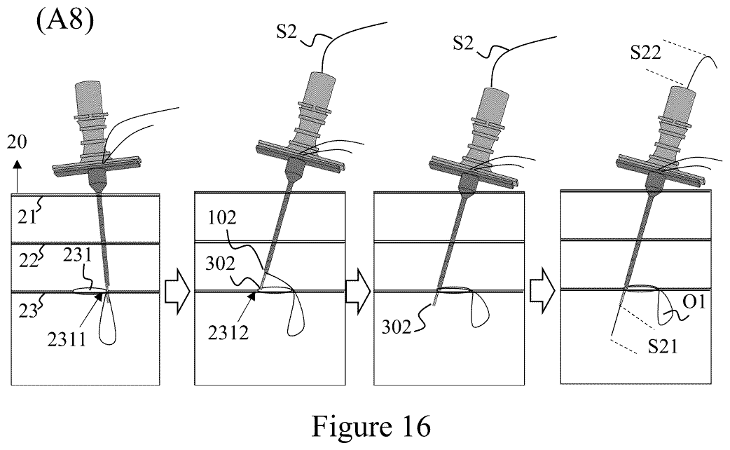

With reference to FIG. 16, step (A8): combining a second suture thread S2 to the inner tubular needle 30, and allowing the second suture thread S2 to reach the lower part of the second side of the hernial orifice 2312: moving the third needle tip portion 302 and the first needle tip portion 102 to the upper part of the second side of the hernial orifice 2312, combining the second suture thread S2 to the inner tubular needle 30, inserting the third needle tip portion 302 into the upper part of the second side of the hernial orifice 2312, thereby allowing the third tip portion to reach the lower part of the second side of the hernial orifice 2312; advancing the second suture thread S2 to allow it to emerge from the third needle tip portion 302 through the second opening 303, wherein the part of the second suture thread S2 that protrudes from the third needle tip portion 302 is defined as a third thread segment, the part of the second suture thread S2 that do not pass through the second opening 303 is defined as a fourth thread segment, herein, the third thread segment reaches the lower part of the second side of the hernial orifice 2312.

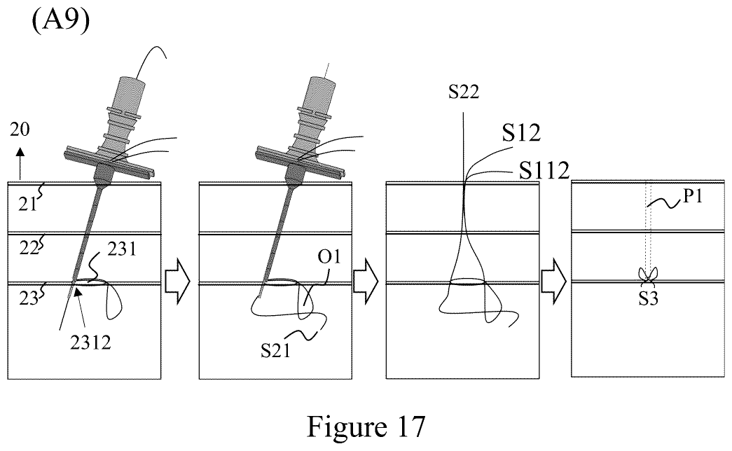

With reference to FIG. 17, step (A9): ligation: in the lower part of the hernial orifice 231, allowing the third thread segment to pass through the first loop O1, maintaining the relative configuration between the third thread segment and the first loop O1, removing the inner tubular needle 30 followed by removing the outer tubular needle 10, lifting the second thread segment S12 and the first outside segment S112 upward to restrict the third thread segment by the first loop O1, forming a first knot S3 by the fourth thread segment, the second thread segment S12, and the first outside segment S112 at the upper part of the body surface 20, and delivering the knot downward through the body surface 20 until it reaches the hernial orifice 231 to close the hernial orifice 231.

With reference to FIG. 17, conceivably, a first suture thread S1 channel, which is formed by the outer tubular needle 10, connects the body surface 20 and the hernial orifice 231, and allows the first knot S3 can be delivered to and close the hernial orifice 231 easily.

Furthermore, as referred in step (A3), the outer tubular needle 10 establishes the first suture thread channel P1 in a single step, wherein the first suture thread channel P1 passes through the fascia layer 22 and the neural layer, therefore, in step (A9), the first knot S3 will not be formed on the fascia layer 22 when closing the hernial orifice 231, and the painless effect can be achieved by preventing the first knot S3 from forming on the neural layer.

As referred in step (A8), based on the inherent relative mobility between the epidermal layer 21, the fascial layer 22, and the peritoneal layer 23, the third needle tip portion 302 and the first needle tip portion 102 can be moved to the upper part of the second side of the hernial orifice 2312 without the requirement of another penetration, accordingly, the first suture thread channel P1 is the only channel needed for delivering the knot formed outside the body surface 20.

With reference to FIG. 11, as referred in step (A3), the first holding portion 203 of the puncturing needle 20 allows the surgeon or the physician to perform the penetration.

With reference to FIG. 11, as referred in step (A3), a side of the first limiting portion 104 relatively farther from the first holding portion 203 can be pressed against the body surface 20, and can avoid excessive insertion of the first opening 103 into the epidermal layer 21, thereby avoiding the operative inconvenience of the outer tubular needle 10.

The following provides a second embodiment of the present invention, including performing (B) a method of painless hernia ligation by using the aforementioned medical needle assembly 1.

Hereinafter, the “upper part”, “upward”, “lower part”, and “downward” referred to herein is used to describe the relative position of a body surface 20 and a hernial orifice 231, for example, is used to describe that the physician performs surgery on the patient in a top-down direction.

Therefore, the body surface 20 refers to a body surface of a patient, “the upper part of the body surface 20” indicates the position outside the patient's body or the position where the physician or the surgeon is located, “the lower part of the body surface 20” indicates the position inside the patient's body or the direction toward the position of the hernial orifice 231.

Furthermore, “the lower part” of the body surface of the patient sequentially comprises: epidermal layer 21, fascial layer 22, peritoneal layer 23, and neural layer between the epidermal layer 21 and the peritoneal layer 23, wherein the hernial orifice 231 is located on the peritoneal layer 23.

It is understood that a first side is located on the opposite side of a second side, with both sides correspondingly formed on the hernial orifice 231.

A method of painless hernia ligation (B) is provided herein, comprising: step (B0), step (B1), and step (B2).
Step (B0): allowing a mesh M to approach an upper part of a hernial orifice 231 or a lower part of the hernial orifice 231, and ensuring the mesh M can completely cover the entire hernial orifice 231, comprising a first side and a second side thereof, after it is attached to the hernial orifice 231. Herein, a first side of the mesh is defined as the side that corresponds to the first side of the hernial orifice 2311, a second side of the mesh is defined as the side that corresponds to the second side of the hernial orifice 2312.
Step (B1): forming a first knot S3 on the first side of the hernial orifice 2311, thereby fixing the first side of the mesh to the first side of the hernial orifice 2311: comprising step (B1) to step (B19):
Step (B11): obtaining a medical needle assembly 1 as mentioned above;
Step (B12): combining the puncturing needle 20 to the outer tubular needle 10: inserting the second needle tip portion 202 into the first opening 103, and allowing the second needle tip portion 202 to protrude from the first needle tip portion 102.
Step (B13): maintaining the outer tubular needle 10 at the location of the body surface 20 corresponding to the hernial orifice 231: using the puncturing needle 20 and the outer tubular needle 10 to penetrate the position on body surface 20 corresponding to the projection of hernial orifice 231 by holding the first holding portion 203, and allowing the second needle tip portion 202 to penetrate a first side of the hernial orifice 2311 and to reach the lower part of the first side; removing the puncturing needle 20 from the first opening 103, maintaining the outer tubular needle 10, and maintaining the first needle tip portion 102 near to the first side of the hernial orifice 2311.
Step (B14): combining the first suture thread S1 to the inner tubular needle 30: inserting the first suture thread S1 into the second opening 303, and protruding the first suture thread S1 from the third needle tip portion 302, wherein the part of the first suture thread S1 protruding from the third needle tip portion 302 is defined as a first thread segment S11, and the part of the first suture thread S1 that does not insert into the inner tubular needle body 301 is defined as a second thread segment S12; and allowing the first thread segment S11 to get close to the second thread segment S12, thereby forming a first inflection point C1 on the first thread segment S11 outside the third needle tip portion 302.
Step (B15): combining the first suture thread S1 and the inner tubular needle 30 to the outer tubular needle 10: inserting the third needle tip portion 302 into the first opening 103 accompanied by the first inflection point C1, and protruding the third needle tip portion 302 from the first needle portion. Understandably, the third needle tip portion 302 is passed through the first side of the hernial orifice 2311, and the first inflection point C1 is reached the lower part of the first side of the hernial orifice 2311. Herein, the first thread segment S11 is further defined as a first inside segment S111, which is extended from the third tip portion and is inserted into the first needle portion, and a first outside segment S112, which is extended from the gap between the first limiting portion 104 and the second holding portion 304 or is extended from the gap the first opening 103 and the second holding portion 304. It is further understood that, under the condition that the outer tubular needle 10 tightly combines to the inner tubular needle 30, which can be referred to that the first limiting portion 104 is tightly combined to the second holding portion 304 or the first opening 103 is tightly combined to the second holding portion 304, the length of the first outside segment S112 is controlled by compression. At this time, if the second thread segment S12 is advanced, the length of first inside segment S111 will increase, and the length of the first outside segment S112 will not increase.
Step (B16): forming a first loop O1 at the lower part of the first side of the hernial orifice 2311: maintaining the tight combination between the first limiting portion 104 and the second holding portion 304 or maintaining the tight combination between the first opening 103 and the second holding portion 304, advancing the second thread segment S12 to increase the length of the first inside segment S111, thereby forming a first loop O1 at the lower part of the first side of the hernial orifice 2311.
Step (B17): removing the inner tubular needle 30 from the outer tubular needle 10, and subsequently recombining the inner tubular needle 30 and the outer tubular needle 10: withdrawing the inner needle through the first opening 103, maintaining the first loop O1 at the lower part of the first side of the hernial orifice 2311, and recombining the inner tubular needle 30 to the outer tubular needle 10, that is to say, the third needle tip portion 302 protrudes from the first needle tip portion 102 through the first opening 103. Herein, the second thread segment S12 and the first outside segment S112 are both extended from the space between the first limiting portion 104 and the second holding portion 304, and the first loop O1 is extended from the first needle tip portion 102.
Step (B18): combining a second suture thread S2 to the inner tubular needle 30, and allowing the second suture thread S2 to reach the lower part of the second side of the hernial orifice 2312: moving the third needle tip portion 302 and the first needle tip portion 102 to the upper part of the second side of the hernial orifice 2312, combining the second suture thread S2 to the inner tubular needle 30, inserting the third needle tip portion 302 into the upper part of the second side of the hernial orifice 2312, thereby allowing the third tip portion to reach the lower part of the second side of the hernial orifice 2312; advancing the second suture thread S2 to allow it to emerge from the third needle tip portion 302 through the second opening 303, wherein the part of the second suture thread S2 that protrudes from the third needle tip portion 302 is defined as a third thread segment, the part of the second suture thread S2 that do not pass through the second opening 303 is defined as a fourth thread segment, herein, the third thread segment reaches the lower part of the second side of the hernial orifice 2312.
Step (B19): a first ligation: in the lower part of the hernial orifice 231, allowing the third thread segment to pass through the first loop O1, maintaining the relative configuration between the third thread segment and the first loop O1, removing the inner tubular needle 30 followed by removing the outer tubular needle 10, lifting the second thread segment S12 and the first outside segment S112 upward to restrict the third thread segment by the first loop O1, forming a first knot S3 by the fourth thread segment, the second thread segment S12, and the first outside segment S112 at the upper part of the body surface 20, and delivering the knot downward through the body surface 20 until it reaches the hernial orifice 231 to close the hernial orifice 231.
Step (B2): forming a second knot S4 on the second side of the hernial orifice 2312, thereby fixing the mesh M to the hernial orifice 231, specifically, step (B2) comprises step (B11) and step (B19). Understandably, as described in the step (B1), the first side of the hernial orifice 2311 is replaced to the second side of the hernial orifice 2312, the first side of the mesh is replaced to the second side of the mesh, therefore the second side of the mesh can be fixed to the second side of the hernial orifice 2312.

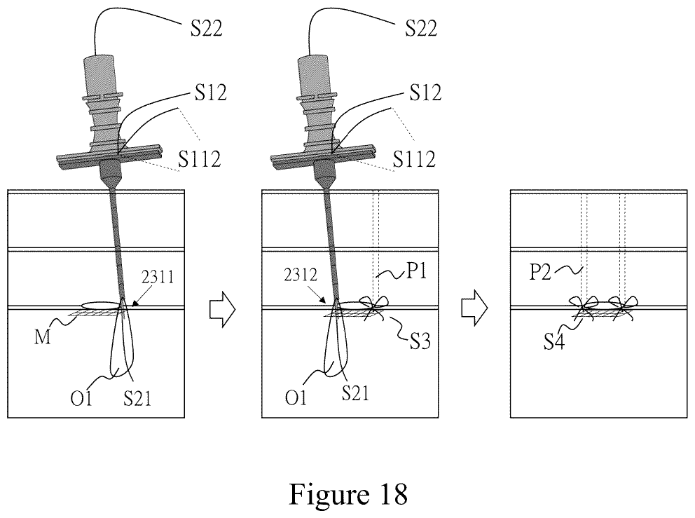

With reference to FIG. 18, FIG. 18 is a schematic diagram of a method of painless hernia ligation using mesh; As illustrated in FIG. 18, the first knot S3 on the first side of the hernial orifice 2311, formed in the step (B1), and the second knot S4 on the second side of the hernial orifice 2312, formed in the step (B2), are configured to fix the mesh M on the hernial orifice 231, respectively.

Comprehensively, a step (B3) is allowed to be further comprised for forming a third knot on a random side of the hernial orifice 231, and is not limited to this.

Accordingly, through step (B1) and step (B2), the hernial orifice 231 can be closed by the mesh M.

Furthermore, as referred in step (B13), the outer tubular needle 10 establishes the first suture thread channel P1 in a single step, wherein the first suture thread channel P1 passes through the fascia layer 22 and the neural layer, therefore, in step (A9), the first knot S3 will not be formed on the fascia layer 22 when closing the hernial orifice 231, and the painless effect can be achieved by preventing the first knot S3 from forming on the neural layer.

In fact, the mesh M can be replaced to any material or element used to fasten, coat, protect, or support, for example, a steel plate.

Furthermore, the number of the suture thread channel is not limited, therefore, in order to reduce the penetration of the outer tubular needle 10 on the body surface 20, correspondingly, the number of the suture thread is changeable, and the part of the body surface 20 that the outer tubular needle 10 penetrates at is changeable. For example, the outer tubular needle 10 may be used to penetrate a central part of the body surface 20 corresponding to the hernial orifice 231, in order to fix the mesh M on the first side and the second side of the hernial orifice 2312.

The following provides a third embodiment of the present invention:

A method for suturing a wound (C), comprising steps as described in the method of painless hernial ligation (A), wherein in the step (A3), the hernial orifice 231 is replace to a random wound, the random wound is located on the body surface 20 or is located inside the body, and is not limited to these.

Through the method for suturing the wound (C), it should be known that, based on the controllability of the puncturing depth of the medical needle assembly 1, the medical needle assembly 1 can be used to suture a wound inside the body, including a hernia, as well as a wound located on the body surface 20.

In fact, the random wound is not limited to a specific tissue, the random wound may involve a hard tissue or a soft tissue, such as connective tissue, mucosal tissue, muscular tissue, adipose tissue, or osseous tissue.

Furthermore, the type of the random wound includes tears or defects, and are not limited to having a considerable degree of openness.

The foregoing description merely illustrates the preferred embodiments of the present invention and should not be construed as limiting the scope of the invention. Any simple equivalent changes or modifications made in accordance with the scope of the patent claims and the description of the invention shall fall within the scope covered by the patent of the present invention.

1. A medical needle assembly, comprising:
an outer tubular needle, comprising:
an outer tubular needle body having a hollow interior extending through both ends, wherein the outer tubular needle body is defined as having a first inner diameter, a first length, and a first opening arranged on an end of the outer tubular needle body;
a puncturing needle, comprising:
a puncturing needle body with both ends having a first outer diameter and a second length; and
an inner tubular needle, comprising:
an inner tubular needle body having a hollow interior extending through both ends, wherein the inner tubular needle body is defined as having a second outer diameter, a third length, and a second opening arranged on an end of the inner tubular needle body;
wherein the first inner diameter is larger than the first outer diameter and the second outer diameter, and the first length is less than the second length and the third length; thereby:
(A) inserting an end of the puncturing needle body into the hollow interior of the outer tubular needle body through the first opening, protruding the end of the puncturing needle body from the other end of the outer tubular needle body to contact a wound, and separating the puncturing needle body and the outer tubular needle body;
(B) inserting an end of a first suture thread into the hollow interior of the inner tubular needle body through the second opening, protruding the end of the first suture thread from the other end of the inner tubular needle body, moving the end of the first suture thread close to the other end of the first suture thread outside the hollow interior of the inner tubular needle body, and forming a first inflection point on a part of the first suture thread near to the other end of the inner tubular needle body; and
(C) maintaining the first inflection point, inserting the other end of the inner tubular needle body into the outer tubular needle body through the first opening, protruding the first inflection point and the other end of the inner tubular needle body from the other end of the outer tubular needle body, and allowing the first inflection point to form a first loop by advancing the other end of the first suture thread.

2. The medical needle assembly as claimed in claim 1, wherein the outer tubular needle further comprises:
a first limiting portion arranged around the first opening; and
a first needle tip portion arranged on the other end of the outer tubular needle body.

3. The medical needle assembly as claimed in claim 1, wherein the puncturing needle further comprises:
a second needle tip portion arranged on an end of the puncturing needle body; and
a first holding portion arranged on the other end of the puncturing needle body.

4. The medical needle assembly as claimed in claim 1, wherein the inner tubular needle further comprises:
a second holding portion arranged around the second opening; and
a third needle tip portion arranged on the other end of the inner tubular needle portion.

5. The medical needle assembly as claimed in claim 1, wherein the first length ranges from 4 to 10 cm, the second length ranges from 5 to 11 cm, and the third length ranges from 6.5 to 12.5 cm.

6. The medical needle assembly as claimed in claim 1, wherein the first length is 7 cm, the second length is 8 cm, and the third length is 9.5 cm.

7. The medical needle assembly as claimed in claim 1, wherein the first inner diameter ranges from 2 to 5 mm, the first outer diameter ranges from 1.5 to 4 mm, the second outer diameter ranges from 1.5 to 3 mm.

8. The medical needle assembly as claimed in claim 1, wherein:
the outer tubular needle further comprises a first limiting portion arranged on the other end of the outer tubular needle body; and
the inner tubular needle further comprises a second holding portion arranged around the second opening.

9. The medical needle assembly as claimed in claim 8, wherein:
the first limiting portion has a first interface arranged between the first limiting portion and the first opening; and
the second holding portion has a second interface arranged between the second holding portion and the second opening.

10. The medical needle assembly as claimed in claim 9, wherein:
an interior of the first interface has a groove structure, an opening of the groove structure is arranged on an end of the first limiting portion relatively distant from the first opening, a bottom of the groove structure connects to the first opening; and
an exterior of the second interface has a protruding structure extending toward the inner tubular needle body, a surface of the protruding structure has a spline groove parallel to the inner tubular needle body.

11. The medical needle assembly as claimed in claim 10, wherein:
the first interface is complementary to the second interface; and
the spline groove is configured to accommodate the first suture thread.

12. A method of treating a wound in a body cavity of a subject, comprising:
step (A): providing a medical needle assembly comprising:
an outer tubular needle, comprising:
an outer tubular needle body having a hollow interior extending through both ends, wherein the outer tubular needle body is defined as having a first inner diameter, a first length, and a first opening arranged on an end of the outer tubular needle body;
a puncturing needle, comprising:
a puncturing needle body with both ends having a first outer diameter and a second length; and
an inner tubular needle, comprising:
an inner tubular needle body having a hollow interior extending through both ends, wherein the inner tubular needle body is defined as having a second outer diameter, a third length, and a second opening arranged on an end of the inner tubular needle body;
wherein the first inner diameter is larger than the first outer diameter and the second outer diameter, and the first length is less than the second length and the third length; and
step (B): treating the wound with the medical needle assembly.

13. The method as claimed in claim 12, wherein the wound is located on a peritoneum in the body cavity.

14. The method as claimed in claim 12, wherein the step (B) further comprises:
establishing a single suture thread channel from a body surface of the subject to the wound using the outer tubular needle and the puncturing needle; and
sending a knot to the wound through the single suture thread channel, thereby closing the wound.

15. The method as claimed in claim 12, wherein the step (B) further comprises:
establishing a single suture thread channel from a body surface of the subject to the wound using the outer tubular needle and the puncturing needle;
allowing an assisting element to contact the wound; and
sending a knot to the wound through the single suture thread channel, thereby binding the assisting element to the wound and closing the wound.

16. The method as claimed in claim 15, wherein the assisting element comprises: fastening element, coating element, protective element, supporting element or any combination thereof.

17. A method of treating a hernia in a subject, comprising:
step (A): providing a medical needle assembly comprising:
an outer tubular needle, comprising:
an outer tubular needle body having a hollow interior extending through both ends, wherein the outer tubular needle body is defined as having a first inner diameter, a first length, and a first opening arranged on an end of the outer tubular needle body;
a puncturing needle, comprising:
a puncturing needle body with both ends having a first outer diameter and a second length; and
an inner tubular needle, comprising:
an inner tubular needle body having a hollow interior extending through both ends, wherein the inner tubular needle body is defined as having a second outer diameter, a third length, and a second opening arranged on an end of the inner tubular needle body;
wherein the first inner diameter is larger than the first outer diameter and the second outer diameter, and the first length is less than the second length and the third length; and
step (B): using the medical needle assembly to introduce a suture thread into the abdomen.

18. The method as claimed in claim 17, wherein the step (B) further comprises:
establishing a single suture thread channel from a body surface of the subject to a wound of the hernia using the outer tubular needle and the puncturing needle; and
sending a knot to the wound through the single suture thread channel, thereby closing the wound.

19. The method as claimed in claim 17, wherein the step (B) further comprises:
establishing a single suture thread channel from a body surface of the subject to a wound of the hernia using the outer tubular needle and the puncturing needle;
allowing an assisting element to contact the wound; and
sending a knot to the wound through the single suture thread channel, thereby binding the assisting element to the wound and closing the wound.

20. The method as claimed in claim 19, wherein the assisting element comprises: fastening element, coating element, protective element, supporting element or any combination thereof.
